(19) United States
(12) Patent Application Publication
(10) Pub. No.: US 2016/0022150 A1
(43) Pub. Date: Jan. 28, 2016

(54) PHOTOACOUSTIC APPARATUS

(71) Applicant: CANON KABUSHIKI KAISHA, Tokyo (JP)

(72) Inventors: Koichiro Wanda, Yokohama-shi (JP); Robert A Kruger, Oriental, NC (US)

(21) Appl. No.: 14/804,013

(22) Filed: Jul. 20, 2015

Related U.S. Application Data
(60) Provisional application No. 62/028,571, filed on Jul. 24, 2014.

(51) Int. Cl. A61B 5/00

(52) U.S. Cl. CPC A61B 5/0095; A61B 5/742

(57) ABSTRACT

A photoacoustic apparatus disclosed in this specification includes a light source; a probe including a plurality of transducers each configured to receive a photoacoustic wave generated from a subject irradiated with light emitted from the light source and output a reception signal, and a support member configured to support the plurality of transducers so that directivity axes of the plurality of transducers are collected; a moving unit configured to move the probe; a region setting unit configured to set an imaging region; and a processing unit configured to acquire subject information in the imaging region based on the reception signals output from the plurality of transducers. The light source is configured to emit the light if a position at which the directivity axes are collected is farther from the probe than a center of the imaging region.

### BACKGROUND OF THE INVENTION

1. Field of the Invention

The present invention relates to a photoacoustic apparatus that acquires subject information by using a photoacoustic effect.

2. Description of the Related Art

Studies of optical imaging apparatuses that each cause light irradiated on a subject from a light source such as a laser to propagate in the subject and acquire information in the subject have been actively advanced with a particular emphasis on the medical field. Photoacoustic Tomography (PAT) has been suggested as one of such optical imaging apparatuses. PAT is a technology that visualizes information relating to optical characteristics of the inside of a subject (in the medical field, living body) by irradiating the subject (living body) with light and receiving and analyzing a photoacoustic wave generated because the light propagating and being diffused in the subject is absorbed by a living body tissue. Accordingly, living body information such as an optical characteristic value distribution in the subject, in particular, an optical energy absorption density distribution can be acquired.

Regarding the information relating to the optical characteristics acquired by this technology, for example, information, such as an initial sound pressure distribution or an optical energy absorption density distribution, generated by the light irradiation can be used for specifying the position of a malignant tumor accompanying with growth of new blood vessels. Generation and displaying of a three-dimensional reconstruction image based on the information relating to the optical characteristics are useful for grasping the inside of a living body tissue, and is expected to help a diagnosis in the medical field.

Japanese Patent Laid-Open No. 2012-179348 describes a plurality of transducers which are fixed to a container having a hemispherical surface and receiving surfaces of which face the center of the hemisphere. Also, referring to Japanese Patent Laid-Open No. 2012-179348, an image obtained by using such a probe has the highest resolution at the center point of the hemisphere and has a high-resolution region near the center point of the hemisphere. Japanese Patent Laid-Open No. 2012-179348 also describes decreasing a variation in resolution by relatively moving the probe and the subject.

However, for the measurement based on the high-resolution region defined in Japanese Patent Laid-Open No. 2012-179348, the resolution in the imaging region is desired to be further increased.

Therefore, this specification provides a photoacoustic apparatus that can acquire subject information in an imaging region with high resolution.

### SUMMARY OF THE INVENTION

A photoacoustic apparatus disclosed in this specification includes a light source; a probe including a plurality of transducers each configured to receive a photoacoustic wave generated from a subject irradiated with light emitted from the light source and output a reception signal, and a support member having an opening and configured to support the plurality of transducers so that directivity axes of the plurality of transducers are collected; a moving unit configured to two-dimensionally move the probe in an in-plane direction of the opening; a region setting unit configured to set an imaging region; and a processing unit configured to acquire subject information in the imaging region based on the reception signals output from the plurality of transducers. The light source is configured to emit the light if a position at which the directivity axes are collected is farther from the probe than a center of the imaging region.

Another photoacoustic apparatus disclosed in this specification includes a light source; a probe including a plurality of transducers each configured to receive a photoacoustic wave generated from a subject irradiated with light emitted from the light source and output a reception signal, and a support member configured to support the plurality of transducers so that directivity axes of the plurality of transducers are collected; a moving unit configured to move the probe; a region setting unit configured to set an imaging region; and a processing unit configured to acquire subject information in the imaging region based on the reception signals output from the plurality of transducers. The light source is configured to emit the light at a plurality of time points. The moving unit is configured to move the probe so that a locus of a region near the probe of a sphere centered on a position at which the directivity axes are collected at the plurality of respective time points fills the imaging region.

Further features of the present invention will become apparent from the following description of exemplary embodiments with reference to the attached drawings.

### BRIEF DESCRIPTION OF THE DRAWINGS

FIGS. 1A and 1B are illustrations showing states of measurements according to a comparative example and a first embodiment.

FIG. 2 is an illustration showing an example of a configuration of a signal measurement unit according to the first embodiment.

FIG. 3 is a functional block diagram of an information processing unit according to the first embodiment.

FIG. 4 is an illustration showing an example of a hardware configuration of the information processing unit according to the first embodiment.

FIG. 5 is a flowchart of an operation of a photoacoustic apparatus according to the first embodiment.

FIGS. 6A and 6B are illustrations each showing an example of a measurement method according to the first embodiment.

FIG. 7 is an illustration showing an example of a measurement method according to a second embodiment.

### DESCRIPTION OF THE EMBODIMENTS

Desirable embodiments of photoacoustic apparatuses according to the present invention are described in detail below with reference to the accompanying drawings. However, the scope of the invention is not limited to the illustrated examples.

### First Embodiment

In a first embodiment, an example of a photoacoustic apparatus that two-dimensionally moves a probe to increase the resolution in an imaging region designated by a user is described.

Regarding the high-resolution region defined in Japanese Patent Laid-Open No. 2012-179348, the resolution tends to be the highest at the center point of the hemisphere and tends to decrease as the distance from the center point of the hemisphere increases. For example, according to Expression (1), a spherical region centered on the center point (curvature center point) of the hemisphere is determined as the high-resolution region.

$${d}_{th} = \frac{{r}_{0}}{{φ}_{d}} \cdot R \qquad ( 1 )$$

In this case, dₜₕ is a radius of the high-resolution region, R is a lower-limit resolution of the high-resolution region, r₀ is a radius of the support member of the hemispherical shape, and φ_{d} is a diameter of the transducers. R can be, for example, a resolution of a half of the highest resolution obtained at the curvature center point.

However, the inventor of the present invention has found that the method using the high-resolution region defined such that the resolution isotropically decreases from the curvature center requires further improvement to increase the resolution in the imaging region.

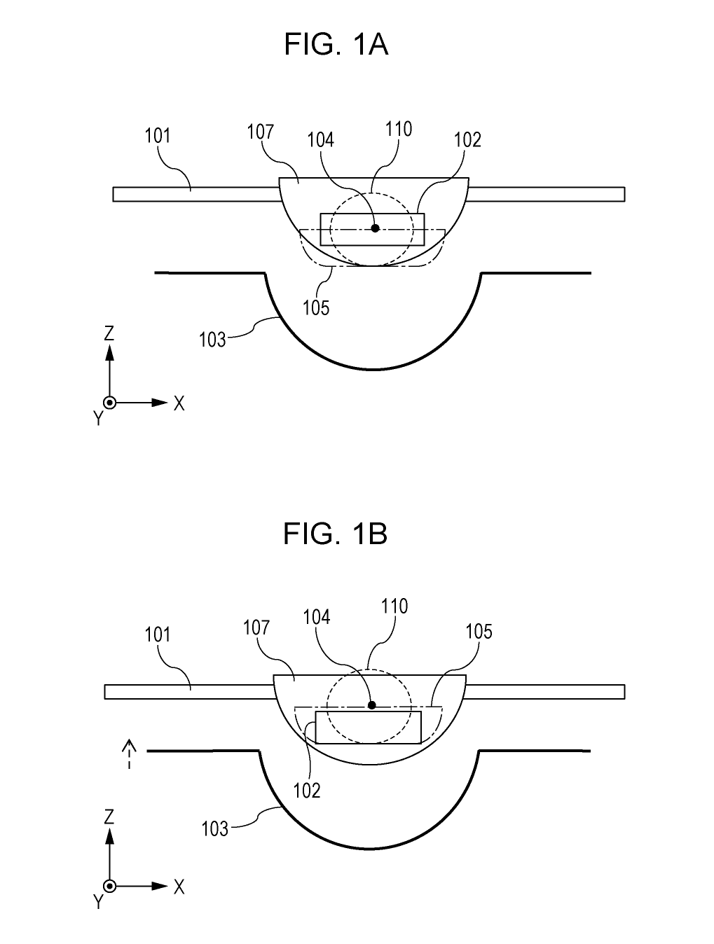

Hereinafter, a comparative example using the high-resolution region defined such that the resolution isotropically decreases from the curvature center, and this embodiment are described below with reference to FIGS. 1A and 1B. In FIGS. 1A and 1B, a bed 101 serving as a subject person support portion is a bed where a subject person lies down. In FIGS. 1A and 1B, to take an image of a breast of a subject person as a subject, the bed 101 has an insertion hole that allows a breast serving as the subject 107 to be inserted. FIGS. 1A and 1B each show a state in which the subject person lies down at a prone position and hence inserts the breast serving as the subject 107 into the insertion hole of the bed 101. An imaging region 102 designated by a user through an input unit is shown.

FIG. 1A illustrates a position of a probe 103 when the resolution in the imaging region 102 is attempted to be increased in accordance with the high-resolution region defined by Expression (1) and having the spherical shape as the comparative example. To increase the resolution in the imaging region 102 in accordance with the high-resolution region defined by Expression (1), a curvature center 104 of the hemisphere may be located on a center plane of the imaging region 102. In the high-resolution region defined by Expression (1), since the resolution isotropically decreases from the curvature center 104 of the probe 103, the position of the probe 103 is desirable because the resolution on the center plane of the imaging region 102 becomes the highest. In this case, the center plane of the imaging region 102 represents a plane that passes through the center of the imaging region 102 and is parallel to an opening of the probe 103. That is, the center plane of the imaging region 102 represents a plane that passes through an intermediate point of the imaging region 102 in the out-plane direction of the opening of the probe 103 and is parallel to the opening of the probe 103.

Meanwhile, it is known that a sound source of a photoacoustic wave can be completely reproduced if a probe surrounds the entire periphery of the sound source, and an artifact, which is generated by reconstruction based on the photoacoustic wave is not generated ideally. That is, it is known that, at a position surrounded by the probe 103, the artifact is reduced and the resolution is increased. It may be conceived that the high-resolution region defined by Expression (1) is defined on the basis of this knowledge. With this knowledge, the high-resolution region is defined as a sphere 110 centered on the curvature center 104 of the probe 103. That is, the high-resolution region in which the resolution isotropically changes is defined.

However, regarding attenuation in photoacoustic wave during propagation, the inventor of the present invention has found that a region with high image quality is different from the high-resolution region having the spherical shape. The attenuation amount of a photoacoustic wave during propagation is smaller as the distance from a generation position of the photoacoustic wave to a transducer is smaller. Hence, if the distance between the generation position of the photoacoustic wave and the transducer is small, S/N of a reception signal of the photoacoustic wave generated at this position is high, and the resolution at this position is high. Owing to this, applying this finding to the sphere 110 centered on the curvature center 104 of the probe 103, the inventor of the present invention has found that a region near the probe 103 of the sphere tends to have higher image quality than a region far from the probe 103 of the sphere. That is, the inventor of the present invention has found that the S/N and resolution are higher in the region near the probe 103 than the region far from the probe 103. Hereinafter, a region near the probe 103 of the sphere 110 centered on the curvature center 104 of the probe 103 is called “measurement region.” In this embodiment, a hemispherical region near the probe 103 included in the region near the probe 103 of the sphere 110 centered on the curvature center 104 is described as a measurement region.

Further, based on the above-described finding, the inventor of the present invention has gotten an idea that the measurement region is moved by moving the probe 103 as shown in FIG. 1B so that the imaging region 102 is filled with a locus 105 of the measurement region. At this time, the probe 103 is moved from the state in FIG. 1A in the out-plane direction (Z direction) of the opening of the probe 103. Accordingly, the curvature center 104 of the probe 103 is located to be farther from the probe 103 than the center plane of the imaging region 102. Then, the probe 103 is moved in the in-plane direction (XY directions) of the opening of the probe 103, the measurement region is moved, and the locus 105 of the measurement region is formed. The locus 105 of the measurement region is obtained by causing measurement regions at light irradiation at a plurality of time points to overlap each other and be joined together.

Accordingly, a photoacoustic wave generated in a measurement region, which is defined with regard to the influence of attenuation during propagation of the photoacoustic wave in addition to the influence of the artifact generated by reconstruction and which has high S/N and resolution, can be effectively received. Meanwhile, in the case of FIG. 1A, as it is understood from that the locus 105 of the measurement region is not arranged within the imaging region 102, the S/N and resolution in the imaging region 102 can be increased according to this embodiment as compared with the comparative example.

Since the artifact generated by reconstruction is restricted at the curvature center 104 in the viewpoint of numerical aperture, it is desirable to measure a photoacoustic wave when the probe 103 is moved so that the curvature center 104 is arranged in the imaging region 102.

Also, the probe 103 more likely receives a photoacoustic wave generated in a region near the probe with respect to the insertion hole provided at the bed 101. Hence, to effectively receive the photoacoustic wave generated in the region near the probe 103 with respect to the insertion hole, it is desirable to measure the photoacoustic wave when the probe 103 is moved so that the curvature center 104 is located near the probe 103 with respect to the insertion hole.

Also, the radius dₜₕ of the sphere 110 centered on the curvature center 104 can be determined by Expression (1). However, if the radius dₜₕ is determined according to Expression (1), it is assumed that the highest resolution is a resolution at the curvature center 104 determined regardless of the attenuation of the photoacoustic wave. Also, the lower-limit resolution R can be set as a value that is a half of the highest resolution.

### Basic Configuration of Photoacoustic Apparatus

The photoacoustic apparatus according to this embodiment can acquire subject information by detecting a photoacoustic wave generated by a photoacoustic effect. The photoacoustic apparatus according to this embodiment is mainly divided into a signal measurement unit 1100 that acquires a reception signal of a photoacoustic wave, and an information processing unit 1000 that acquires subject information based on the reception signal.

In this embodiment, the subject information is, for example, an initial sound pressure of a photoacoustic wave, an optical energy absorption density derived from the initial sound pressure, an absorption coefficient, a density of a substance configuring a subject, etc. In this case, the density of a substance is an oxygen saturation, an oxyhemoglobin density, a deoxyhemoglobin density, a total hemoglobin density, etc. The total hemoglobin density is the sum of the oxyhemoglobin density and the deoxyhemoglobin density.

Also, in this embodiment, the subject information may not be numerical data and may be distribution information at each position in a subject. That is, distribution information, such as an absorption coefficient distribution or an oxygen saturation distribution, may serve as the subject information.

### Basic Configuration of Signal Measurement Unit 1100

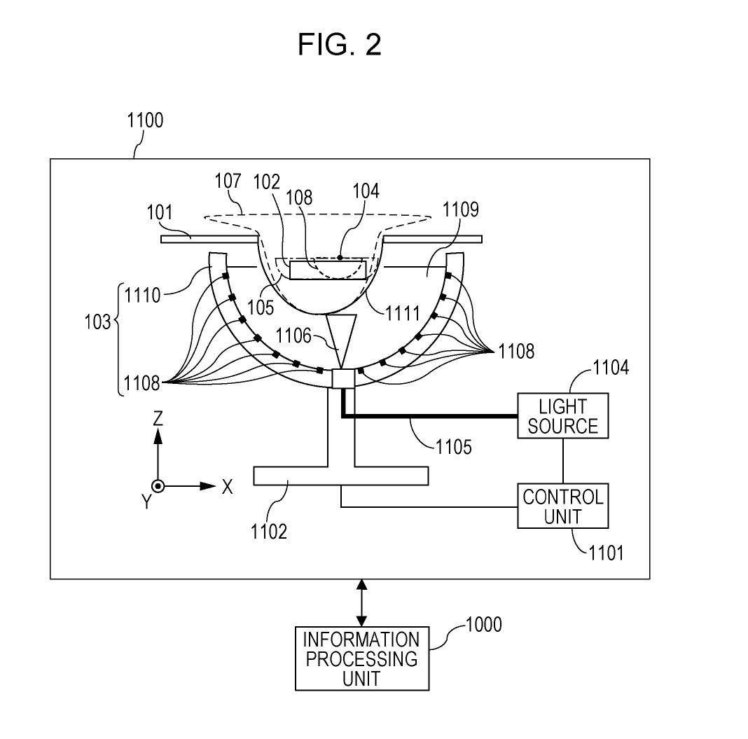

FIG. 2 is an illustration showing an example of a configuration of the signal measurement unit 1100 of the photoacoustic apparatus according to the embodiment of the present invention.

The signal measurement unit 1100 is a block that measures a signal of a photoacoustic wave in the embodiment of the present invention. The signal measurement unit 1100 includes a control unit 1101, a moving unit 1102, the probe 103, a light source 1104, and an optical system 1105.

First, light emitted from the light source 1104 is irradiated on the subject 107, as pulsed light 1106 through the optical system 1105. Then, a photoacoustic wave is generated in the subject 107 by a photoacoustic effect. Then, the propagating photoacoustic wave is received by the probe 103; and an electrical signal on time-series is acquired, stored in the information processing unit 1000, and serves as reception signal data.

Also, the above-described process is executed while the position of the probe 103 is changed by the moving unit 1102, so that the reception signal data is generated at each of a plurality of measurement positions. In this case, the measurement position represents a position at which the probe 103 is located when the subject 107 is irradiated with the pulsed light 1106. Also, positions at which the probe 103 is located at the respective time points when the subject 107 is irradiated with the pulsed light 1106 at a plurality of time points are collectively called “a plurality of measurement positions.”

Next, the information processing unit 1000 acquires the subject information in the imaging region set on the basis of the reception signal data, and causes a displaying unit of the information processing unit 1000 to display the subject information.

### Control Unit 1101

The control unit 1101 controls respective configurations of the signal measurement unit 1100 including the moving unit 1102, the probe 103, the light source 1104, and the optical system 1105. The control unit 1101 is typically configured of a CPU.

The control unit 1101 causes the probe 103 to perform scanning by using the moving unit 1102. Also, the control unit 1101 controls the light source 1104 and the optical system 1105, and hence the subject 107 is irradiated with the pulsed light 1106 and a photoacoustic wave is detected through the probe 103.

The control unit 1101 amplifies an electrical signal of the photoacoustic wave acquired through a transducer 1108 of the probe 103, and converts the signal from an analog signal into a digital signal. Also, various signal processing and various correction processing are executed. Further, a photoacoustic wave signal is transmitted from the signal measurement unit 1100 to an external device, for example, the information processing unit 1000 through an interface (not shown).

Alternatively, the information processing unit 1000 and the control unit 1101 may be integrally configured. That is, the function of the control unit 1101 may be realized by the information processing unit 1000.

### Moving Unit 1102

The moving unit 1102 relatively moves the subject 107 and the probe 103 in accordance with a control signal from the control unit 1101. For example, the moving unit 1102 is a three-axis stage movable in the Z direction in addition to the XY plane. The moving unit 1102 three-dimensionally changes the relative position of the probe 103 with respect to the subject 107 and performs movement for photoacoustic wave measurement. As the moving method, any moving method may be employed as long as the movement is available in the imaging region instructed by an image taking person. As an example moving method, the probe 103 may be moved in a spiral form.

### Probe 103

The probe 103 includes transducers 1108 and a hemispherical-shaped support member 1110 that supports the transducers 1108. The transducers 1108 are arranged to contact a solution that forms a matching layer 1109 and to surround the subject 107. The transducers 1108 each receive a photoacoustic wave and output an electrical signal as a reception signal on time-series. The transducers 1108 that receive photoacoustic waves from a subject each may use a configuration having high sensitivity and a wide frequency band. To be specific, a transducer using PZT, PVDF, cMUT, or a Fabry-Perot interferometer may be exemplified. However, any configuration may be applied without limiting to the above-described configuration as long as the configuration can detect a photoacoustic wave.

In general, a transducer has the highest reception sensitivity in the normal line direction to the reception surface (surface) of the transducer. Since the plurality of transducers 1108 are arranged at the hemispherical surface of the hemispherical-shaped support member 1110, axes (hereinafter, referred to as directivity axes) extending along a direction of the highest reception sensitivity of the plurality of transducers 1108 can be collected near the curvature center point of the hemispherical shape. Accordingly, a region available for visualization with high accuracy (high-resolution region) is formed near the curvature center point.

FIG. 2 is an example of the transducer arrangement, and the way of arrangement is not limited thereto. Any way of arrangement of the transducers may be employed as long as the directivity axes are collected in a desirable region and a desirable high-resolution region can be formed. That is, the plurality of transducers 1108 may be arranged along a curved surface shape so that a desirable high-resolution region is formed. Further, in this specification, a curved surface includes a spherical surface having a spherical shape, a hemispherical shape, or the like, with an opening. Also, a surface with surface unevenness to a certain degree that can be recognized as a spherical surface, or a surface on an elliptic body (a shape obtained by extending an ellipse three dimensionally, the surface of the shape being formed of a quadratic surface) to a degree that can be recognized as a spherical surface may be included.

Also, if the plurality of transducers 1108 are arranged along the support member 1110 with a shape obtained by cutting a sphere at a desirable cross section, the directivity axes are collected the most at the curvature center of the shape of the support member. In this specification, a spherical shape obtained by cutting a sphere at a desirable cross section and having an opening is called a shape based on a sphere. Also, the plurality of transducers supported by the support member having the shape based on the sphere are supported on the spherical surface. The hemispherical-shaped support member 1110 described in the embodiment is also an example of the spherical-shaped support member obtained by cutting the sphere at the desirable cross section and having the opening.

The support member 1110 may be configured by using a metal material with a high mechanical strength.

### Light Source 1104

The light source 1104 is a light source having a power sufficient for photoacoustic wave measurement and can change the wavelength if required, for example, a device such as a laser or a light-emitting diode that generates pulsed light. Regarding the wavelength of pulsed light, a light source that can select a wavelength with a high absorption coefficient for an observation object and that can provide irradiation with light in a sufficiently short period of time in accordance with heat characteristics of a subject is used. To be specific, the light source 1104 may generate light with a pulse width of about 10 nanoseconds to efficiently generate a photoacoustic wave. The wavelength of light that can be emitted by the light source 1104 may be a wavelength with which light propagates to the inside of the subject. To be specific, if the subject is a living body, a desirable wavelength is in a range from 500 nm to 1200 nm. When the optical characteristic value distribution of a living tissue located relatively near the surface of the living body is obtained, a wavelength range from 400 nm to 1600 nm, the range which is wider than the above-described wavelength range, may be used.

The laser used as the light source 1104 may be any of various lasers, such as a solid laser, a gas laser, a dye laser, and a semiconductor laser. For example, an alexandrite laser, an Yttrium-Aluminium-Garnet laser, or a Titan-Sapphire laser may be used as the light source 1104.

### Optical System 1105

The optical system 1105 is a device relating to an optical path for guiding light emitted by the light source 1104 to the subject 107 and irradiation of the light. The optical system 1105 may guide the light by using a mirror, an optical fiber, etc., and is constructed by combining optical devices, such as a lens, a filter, a prism, and a diffusing plate. However, as long as a similar function is attained, the optical system 1105 may be configured of other device without limiting to a general optical device. The pulsed light 1106 in FIG. 2 represents light emitted by the light source 1104, guided by the optical system 1105, output from a bottom portion of the probe 103, transmitted through the matching layer 1109, and irradiated on the subject 107.

The laser irradiation time point, waveform, intensity, etc., of light source 1104 and the optical system 1105 are controlled by the control unit 1101. Also, when signal measurement of a photoacoustic wave is performed during imaging, by moving the position of the probe 103 to a proper position by the moving unit 1102, the optical system 1105 is synchronously moved. Also, the control unit 1101 executes respective control for measuring a signal of a photoacoustic wave detected by the probe 103 in synchronization with the time point of laser irradiation. Further, the control unit 1101 may execute signal processing of adding signals obtained from an element at the same position by irradiating the element with a laser beam a plurality of times, obtaining the average of the sum, and thus calculating the average value of the signals at the position. However, when the moving unit 1102 moves the probe 103, a transducer different from the transducer after measurement may occasionally receive a photoacoustic wave at the same position. In this case, since a photoacoustic wave generated at a different position of the subject is acquired due to a difference in directivity, mounting angle, etc., of the element of the transducer 1108, the summation may not be executed. The control unit 1101 transmits signal information to the information processing unit 1000 based on the photoacoustic wave detected by the probe 103.

In this case, the signal information includes the reception signal on time-series output from each transducer 1108. Also, the signal information may include information of the probe 103, such as information relating to the position of the element arranged on the reception surface of the probe 103 and information relating to the sensitivity and directivity. Also, the signal information may include information relating to conditions during signal acquisition of the photoacoustic wave, such as imaging instruction information designated by a user and measurement method information used for operation control of the photoacoustic apparatus. Also, if the photoacoustic wave is received while the probe 103 is moved, the signal information may include information that can specify the position at which the reception signal output from each transducer 1108 at each time point is received. For example, the received position of the photoacoustic wave can be specified by using the three-dimensional coordinate position of the support member 1110 at each time point and arrangement information of the transducers on the support member 1110.

### Subject 107

Although the subject 107 does not configure a portion of the photoacoustic apparatus according to the embodiment of the present invention, the subject 107 is described below. For convenience, the subject 107 is indicated by a broken line in FIG. 2. The photoacoustic apparatus according to this embodiment is provided mainly for a diagnosis for a malignant tumor, a blood vessel disease, etc., of a human or an animal; or follow-up observation etc. of a chemical treatment. Therefore, the subject is expected to be a living body, or more particularly, an object portion for a diagnosis, such as a breast, a neck portion, or an abdominal portion of a human body or an animal.

Also, it is assumed that an optical absorbent in the subject is a substance with a relatively high optical absorption coefficient in the subject. For example, if a human body is a measurement object, oxyhemoglobin or deoxyhemoglobin; a blood vessel containing these by a large amount; or a malignant tumor containing many new blood vessels may be an object of the optical absorbent. In addition, plaque at a carotid artery wall may be also an object.

### Holding Unit 1111

A holding unit 1111 is a member for holding the shape of the subject 107 to be constant. The holding unit 1111 is mounted to the bed 101 serving as a mounting portion. If a plurality of holding units are used for holding the subject 107 respectively in a plurality of shapes, the bed 101 serving as the mounting portion may be configured to allow the plurality of holding units to be mounted.

When the subject 107 is irradiated with light through the holding unit 1111, the holding unit 1111 may be transparent to the irradiation light. For example, the material of the holding unit 1111 may use polymethylpentene or polyethylene terephthalate.

Also, when the subject 107 is a breast, to hold the breast so that deformation of the breast shape is decreased and the shape is held constant, the shape of the holding unit 1111 may be a shape obtained by cutting a sphere at a certain cross section. The shape of the holding unit 1111 may be properly designed in accordance with the volume of a subject and the desirable shape of the subject after the subject is held. The holding unit 1111 may be configured such that the holding unit 1111 is fitted to the outer shape of the subject 107 and the shape of the subject 107 becomes substantially the same as the shape of the holding unit 1111. Alternatively, the photoacoustic apparatus may measure a photoacoustic wave without using the holding unit 1111.

### Matching Layer 1109

The matching layer 1109 is an impedance matching member that fills the space between the subject 107 and the probe 103 to photoacoustically couple the subject 107 with the probe 103. The material may be liquid that has a photoacoustic impedance similar to those of the subject 107 and the transducer 1108, and transmits pulsed light. To be specific, water, castor oil, gel, etc., is used. As described later, since the relative positions of the subject 107 and the probe 103 are changed, both the subject 107 and the probe 103 may be arranged in a solution forming the matching layer 1109.

### Functional Block Diagram of Information Processing Unit 1000

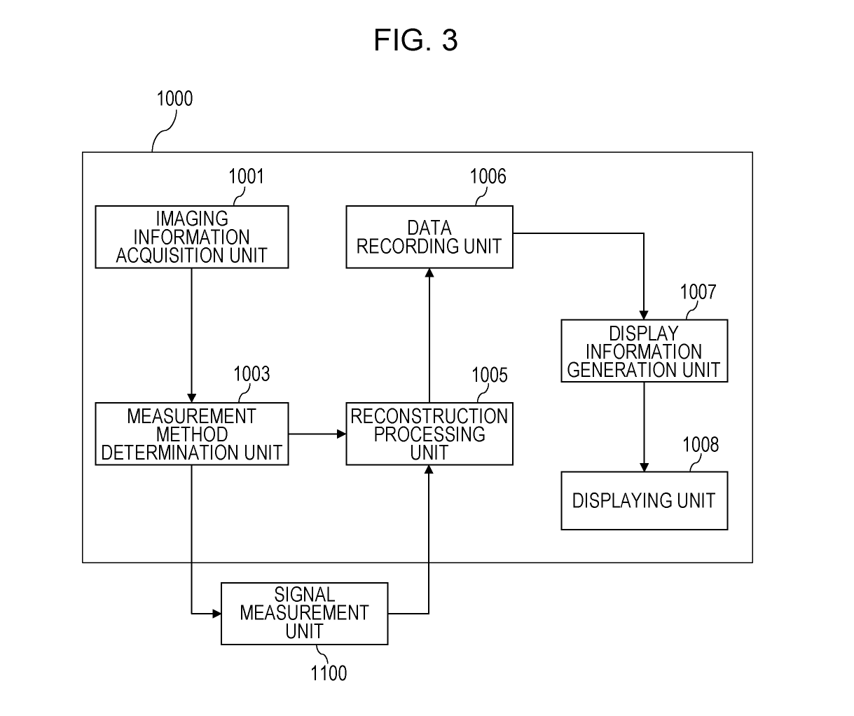

Next, functions of the information processing unit 1000 are described below. FIG. 3 is a functional block diagram showing a functional configuration of the information processing unit 1000 according to this embodiment.

The information processing unit 1000 is configured of an imaging information acquisition unit 1001, a measurement method determination unit 1003, a reconstruction processing unit 1005, a data recording unit 1006, a display information generation unit 1007, and a displaying unit 1008.

### Imaging Information Acquisition Unit 1001

The imaging information acquisition unit 1001 acquires information of an instruction relating to imaging input through an input unit by a user. Then, the imaging information acquisition unit 1001 transmits the information of the instruction relating to imaging as imaging instruction information to the measurement method determination unit 1003.

The information of the instruction relating to imaging represents any kind of instruction relating to imaging that can be input through the input unit by the user. Particularly in this embodiment, described as an example of the information of the instruction relating to imaging is a case in which information relating to an imaging region, which is a region that subject information is finally acquired, is designated by the user with use of the input unit. In this embodiment, the imaging region is a two-dimensional or three-dimensional region. Any method can be employed as long as the method can designate the imaging region.

Also, as the imaging instruction information, the type of moving method of the probe 103 such as linear scanning or spiral scanning, the moving pitch, the number of measurement points, etc., may be instructed in addition to the imaging region. Also, as the imaging instruction information, information relating to a reconstruction processing method and a data saving method after the measurement of the photoacoustic wave may be instructed.

### Measurement Method Determination Unit 1003

The measurement method determination unit 1003 determines a measurement method of the signal measurement unit 1100 based on the imaging instruction information received from the imaging information acquisition unit 1001. That is, the measurement method determination unit 1003 determines an operation method of each configuration of the signal measurement unit 1100 based on the imaging instruction information. The measurement method determination unit 1003 generates information relating to a measurement method, which is a parameter required for an operation performed by each configuration of the signal measurement unit 1100, and transmits the generated information to the signal measurement unit 1100. For example, the measurement method determination unit 1003 can calculate the coordinates of the probe 103 when each pulsed light 1106 is emitted based on the information relating to the imaging region transmitted from the imaging information acquisition unit 1001, as measurement method information. Also, the measurement method determination unit 1003 determines a parameter required for the reconstruction processing unit 1005 based on the imaging instruction information, and transmits a reconstruction parameter as the measurement method information to the reconstruction processing unit 1005. For example, the measurement method determination unit 1003 can determine a region that should be reconstructed by the reconstruction processing unit 1005 based on the information of the imaging region, and can transmit information of a reconstruction region to the reconstruction processing unit 1005.

Alternatively, the measurement method determination unit 1003 may acquire the measurement method information by reading a parameter corresponding to the imaging instruction information acquired by the imaging information acquisition unit 1001 from a memory that stores the parameter based on the imaging instruction information.

Also, the measurement method determination unit 1003 may acquire previously set measurement method information in addition to the acquisition of the measurement method information based on the imaging instruction information designated through the input unit by an image taking person every image taking.

### Reconstruction Processing Unit 1005

The reconstruction processing unit 1005 executes reconstruction processing based on signal information of a photoacoustic wave received from the signal measurement unit 1100, and acquires reconstruction data relating to subject information. Also, the reconstruction processing unit 1005 can execute the reconstruction processing also based on measurement instruction information indicative of measurement conditions of the signal measurement unit 1100. The reconstruction processing unit 1005 executes three-dimensional reconstruction processing by using signal information of a selected photoacoustic wave at each point in an imaging region acquired by the imaging information acquisition unit 1001, and generates three-dimensional reconstruction data (volume data) based on the signal information of the photoacoustic wave. Alternatively, the reconstruction processing unit 1005 may generate two-dimensional reconstruction data (pixel data) without limiting to the three-dimensional reconstruction data, in accordance with the dimension of the imaging region.

The reconstruction processing unit 1005 can reconstruct a photoacoustic wave distribution (initial sound pressure distribution) at light irradiation as reconstruction data based on the signal information of the photoacoustic wave. Also, by using a phenomenon that the degree of absorption of light in a subject is different in accordance with the wavelength of irradiation light, a density distribution of a substance in a subject can be acquired as reconstruction data from an absorption coefficient distribution corresponding to a plurality of wavelengths.

The reconstruction method may be, for example, a UBP method (Universal Backprojection method), a filtered backprojection method, or an iterative reconstruction method. The present invention may use any reconstruction method.

Also, the reconstruction processing unit 1005 can calculate a value indicative of an absorption coefficient distribution in a subject by dividing the reconstructed initial sound pressure distribution by a light fluence distribution in the subject of light irradiated on the subject. Also, by using the phenomenon that the degree of absorption of light in a subject is different in accordance with the wavelength of irradiation light, the reconstruction processing unit 1005 can acquire a density distribution of a substance in a subject as reconstruction data from an absorption coefficient distribution corresponding to a plurality of wavelengths. For example, the reconstruction processing unit 1005 can acquire an oxygen saturation distribution as reconstruction data, for a density distribution of a substance in a subject.

The reconstruction processing unit 1005 transmits the generated reconstruction data to the data recording unit 1006. Additionally, the reconstruction processing unit 1005 may also transmit the imaging instruction information, measurement method information, signal information of the photoacoustic wave, and other information to the data recording unit 1006. However, if the reconstruction data is immediately displayed regardless of whether the data is recorded or not, the reconstruction data may be transmitted to the display information generation unit 1007.

### Data Recording Unit 1006

The data recording unit 1006 saves record data based on the reconstruction data, imaging instruction information, measurement instruction information, reception signal data of the photoacoustic wave, and other data received from the reconstruction processing unit 1005.

For example, volume data obtained by dividing a voxel space corresponding to an imaging region by a pitch determined by setting of reconstruction processing into voxels is saved as record data in which information is added in a data format storing a reconstruction image. Data may be recorded in any data format. For example, volume data can be saved in a format of DICOM (Digital Imaging and Communications in Medicine) being a standard format for medical images. Information relating to the photoacoustic apparatus is stored in a private tag, so that the information can be saved while versatility of DICOM of other information is kept. Also, if data obtained by a plurality of measurements is saved, identifiers for identifying the plurality of measurements are stored in the private tag, and hence respective pieces of reconstruction data of the measurements can be identified.

Also, the data recording unit 1006 may save information included in the signal information of the photoacoustic wave acquired from the signal measurement unit 1100 in any format.

The data recording unit 1006 saves generated data as a record data file in, for example, an auxiliary memory 303 such as a magnetic disk. Alternatively, data may be stored in other information processing apparatus or a computer-readable storage medium through a network, as the data recording unit 1006. Any storage medium can be applied as the data recording unit 1006 as long as the storage medium can save record data.

### Display Information Generation Unit 1007

The display information generation unit 1007 generates display information based on the reconstruction data received from the reconstruction processing unit 1005 or the data recording unit 1006. If the reconstruction data is two-dimensional data and is in a value range that can be directly displayed with luminance values of a display, the display information generation unit 1007 can generate the display information without special conversion. If the reconstruction data is three-dimensional volume data, the display information generation unit 1007 can generate display information by any method, such as volume rendering, a multi-cross-section conversion display method, or a maximum intensity projection (MIP) method. Also, if the value range of the reconstruction data is a value range exceeding the value range of luminance values of the display, the display information generation unit 1007 can execute window processing and generate display information with pixel values that can be displayed on the displaying unit 1008. Also, the display information generation unit 1007 may generate display information in which a plurality of pieces of information are integrated to display the reconstruction data simultaneously with other information.

### Displaying Unit 1008

The displaying unit 1008 is a displaying device, such as a graphic card, a liquid crystal display, or a CRT display, for displaying the generated display information, and displays the display information received from the display information generation unit 1007. Alternatively, the displaying unit 1008 may be provided separately from the photoacoustic apparatus according to this embodiment.

### Hardware Configuration of Information Processing Unit 1000

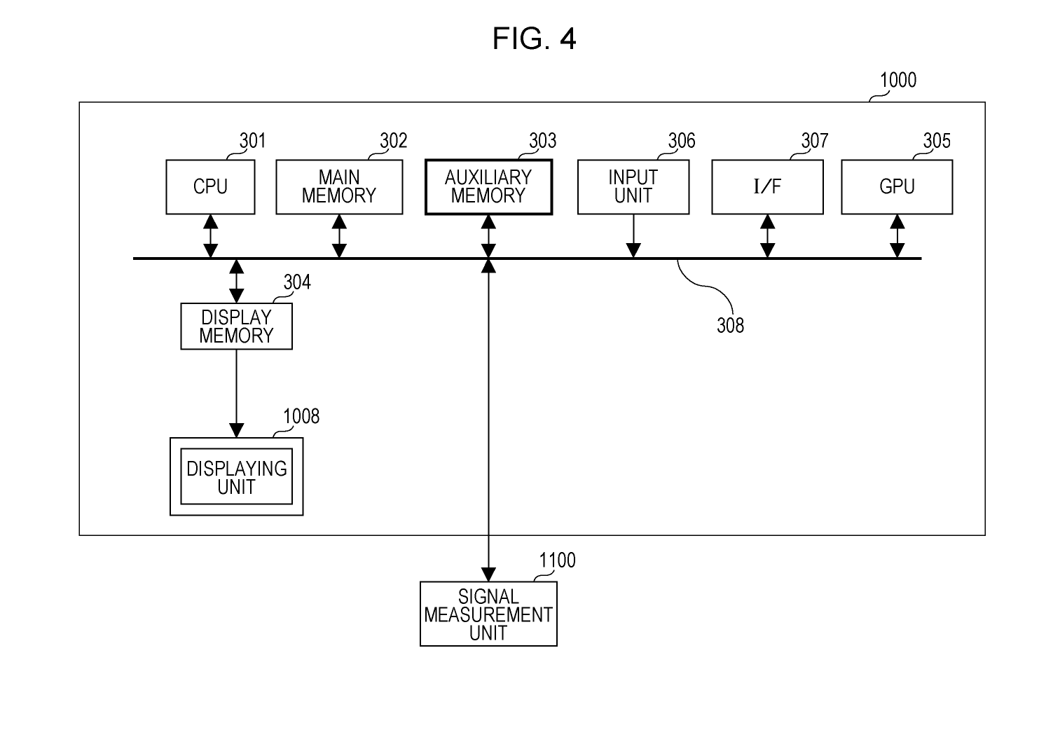

FIG. 4 is an illustration showing a basic configuration of a computer for realizing the functions of the respective units of the information processing unit 1000 by software.

A CPU 301 mainly controls operations of respective components of the information processing unit 1000. A main memory 302 stores a control program that is executed by the CPU 301 and provides a work area during execution of the program by the CPU 301. A semiconductor memory or the like may be used for the main memory 302. In this embodiment, the functions of the imaging information acquisition unit 1001 and the measurement method determination unit 1003 are mainly realized by the CPU 301 and the main memory 302.

The auxiliary memory 303 stores an operating system (OS), a device driver of a peripheral device, and various application software including a program for executing processing of a flowchart (described later), etc. A magnetic disk, a semiconductor memory, or the like may be used for the auxiliary memory 303. A display memory 304 temporarily stores display data for the displaying unit 1008. A semiconductor memory or the like may be used for the display memory 304. In this embodiment, the function of the data recording unit 1006 is realized mainly by the auxiliary memory 303 and the display memory 304.

A GPU 305 executes processing of generating an image of the subject information from the signal information acquired by the signal measurement unit 1100. In this embodiment, the functions of the reconstruction processing unit 1005 and the display information generation unit 1007 are mainly realized by the GPU 305.

An input unit 306 is used for pointing input or input of a character etc. by a user. A mouse, a keyboard, etc., is used for the input unit 306. An operation by a user in this embodiment is performed through the input unit 306.

An I/F 307 is for exchanging various data between the information processing unit 1000 and an external device, and is configured under IEEE1394, US5, or the like. Data acquired through the I/F 307 is taken in the main memory 302.

Operation control of each configuration of the signal measurement unit 1100 is realized through the I/F 307. The above-described components are connected to each other by a common bus 308 in a manner that the components can make communication with each other.

### Operation of Photoacoustic Apparatus

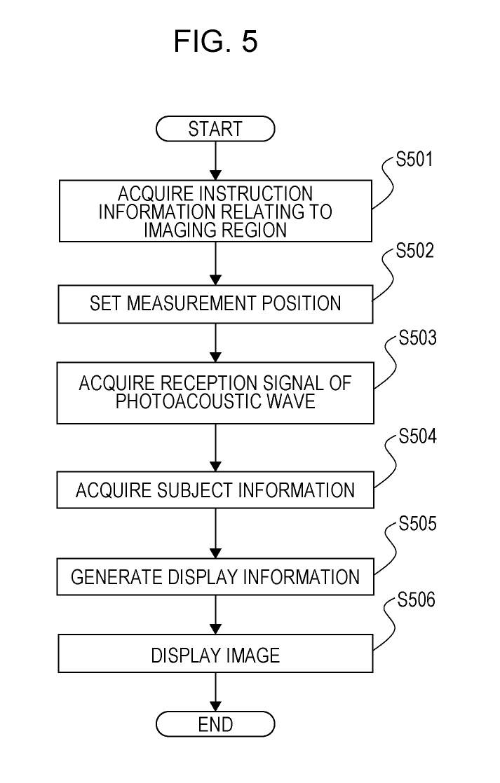

Next, an operation of the photoacoustic apparatus shown in FIG. 2 is described. The flowchart in FIG. 5 is a flowchart for showing the operation of the photoacoustic apparatus according to this embodiment.

### Step S501: Process of Acquiring Instruction Information Relating to Imaging Region

In this process, the imaging information acquisition unit 1001 generates imaging instruction information relating to an imaging region in response to an imaging instruction from a user. The imaging information acquisition unit 1001 transmits the generated imaging instruction information to the measurement method determination unit 1003.

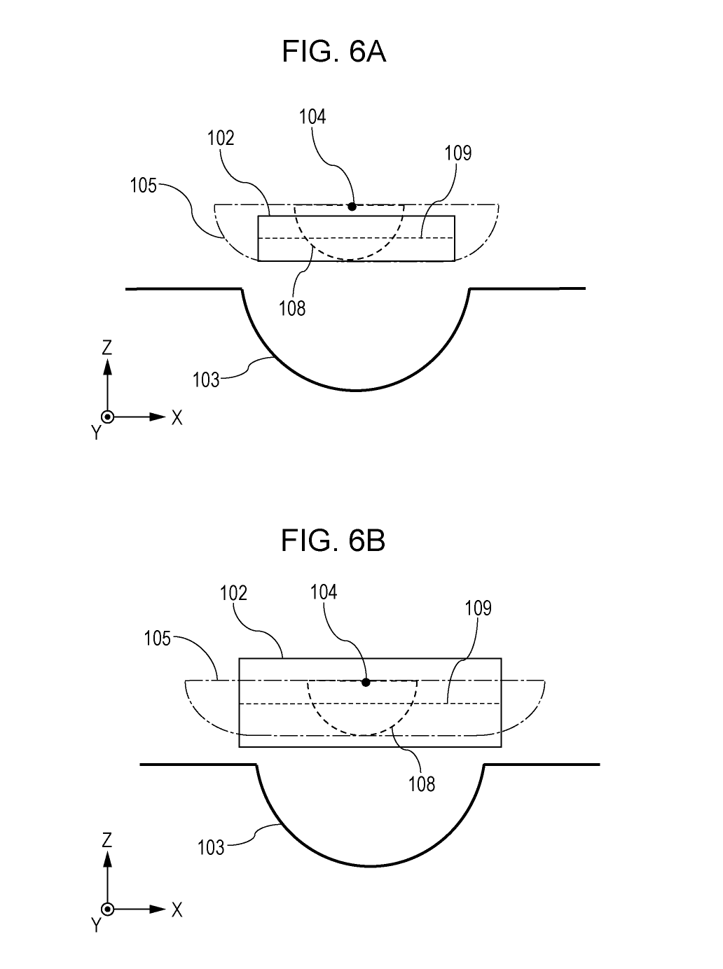

As shown in FIGS. 6A and 6B, the user designates the imaging region 102 as the imaging instruction information through the input unit 306. For example, the information relating to the imaging region may be designated such that the user designates a desirable imaging region by using the input unit 306 from a plurality of previously set imaging regions.

Alternatively, the imaging information acquisition unit 1001 serving as a region setting unit can set the imaging region 102 such that the user inputs the size or position of a three-dimensional region of a predetermined shape by using the input unit 306. Alternatively, the position of the three-dimensional region may be previously set at a position at which a subject is held by the holding unit 1111. Alternatively, the imaging region may be designated by the user by adding an image pickup apparatus such as a video camera (not shown) to the configuration, displaying a rectangular graphic or the like indicative of a camera image capturing a subject and an imaging region, and operating the graphic by using the input unit 306. That is, the input unit 306 is configured such that the user can input the information relating to the imaging region. As long as the imaging region can be designated, the input unit 306 may be configured to allow information relating to any imaging region to be input.

The imaging region may be a region containing the entire subject 107, or the region of a portion of the subject 107 may serve as an imaging region in a limited manner.

### Step S502: Process of Setting Measurement Position

In this process, the measurement method determination unit 1003 sets a measurement position of a photoacoustic wave based on the imaging instruction information relating to the imaging region. That is, the measurement method determination unit 1003 sets the position of the probe 103 at a light irradiation time point, based on the set imaging region 102.

As shown in FIG. 6A, the measurement method determination unit 1003 sets a measurement position so that the measurement region 108 overlaps the imaging region 102 at light irradiation. In FIG. 6A, a transducer is not illustrated for convenience; however, a case in which transducers are arranged on a hemisphere of the probe 103 is considered. In this embodiment, a hemispherical region near the probe 103 of a sphere centered on the curvature center 104 of the probe 103 serves as the measurement region 108. That is, the measurement method determination unit 1003 sets the position of the probe 103 so that the curvature center 104 of the probe 103 is farther from the probe 103 than a center plane 109 of the imaging region 102. Regarding a photoacoustic wave generated in a region near the probe 103 in the measurement region 108, the attenuation occurring until the photoacoustic wave reaches the probe 103 is small. Hence, the resolution in the region tends to be high. Owing to this, if the imaging region 102 is small relative to the measurement region 108, the probe 103 may be positioned so that an end portion near the probe 103 of the measurement region 108 is aligned with an end portion of the imaging region 102. Also, the measurement method determination unit 1003 sets a plurality of measurement positions so that the locus 105 of the measurement region, in which the measurement regions 108 at the plurality of respective light irradiation time points overlap each other and are joined together, fills the imaging region 102. By setting the plurality of measurement positions as described above, the measurement method determination unit 1003 can increase the resolution in the imaging region 102 and decrease a variation in resolution.

Also, like the case in FIG. 6B, a case in which the imaging region is large relative to the measurement region 108 is considered. In this case, the measurement method determination unit 1003 may set the measurement positions so that the measurement regions 108 are positioned in the imaging region 102 as many as possible. That is, the measurement method determination unit 1003 may set the measurement positions so that the measurement region 108 is arranged within the imaging region 102. Hence, the measurement method determination unit 1003 may set the measurement positions so that an end portion near the probe 103 of the measurement region 108 is farther from the probe 103 than an end portion of the imaging region 102 and the curvature center 104 is arranged within the imaging region 102. Also, the measurement method determination unit 1003 may set a plurality of measurement positions so that the locus 105 of the measurement region overlaps the imaging region 102 as much as possible.

The measurement method determination unit 1003 generates measurement method information for controlling the operation of each configuration of the signal measurement unit 1100 so as to attain the above-described measurement positions, and transmits the measurement method information to the signal measurement unit 1100. For example, the measurement method determination unit 1003 generates measurement method information relating to irradiation light control of the signal measurement unit 1100 and the position of the probe 103 moved by the moving unit 1102.

### Step S503: Process of Acquiring Reception Signal of Photoacoustic Wave

In this process, the control unit 1101 of the signal measurement unit 1100 acquires the reception signal of the photoacoustic wave by controlling the respective configurations of the signal measurement unit 1100 based on the measurement method information from the measurement method determination unit 1003.

The moving unit 1102 moves the probe 103 to be at a set measurement position, and the light source 1104 emits light when the probe 103 is positioned at the set measurement position. The pulsed light 1106 is emitted from the light source 1104 to the subject 107 through the optical system 1105, and a photoacoustic wave is generated at the subject 107. The generated photoacoustic wave is received by each transducer 1108, and a reception signal on time-series is output. The reception signal on time-series output from each transducer 1108 is saved as reception signal data acquired at the measurement position set by the information processing unit 1000. Also, information used for measurement of the photoacoustic wave, such as the moving method of the probe 103, the position of the probe 103, and the control method of light irradiation, may be saved in the information processing unit 1000 together with the reception signal data.

### Step S504: Process of Acquiring Subject Information

In this process, the reconstruction processing unit 1005 of the information processing unit 1000 acquires the reconstruction data relating to the subject information in the imaging region 102 set in step S502 based on the reception signal data. The reconstruction processing unit 1005 may acquire the reconstruction data relating to the subject information in the imaging region 102 also based on the information used for the measurement of the photoacoustic wave in addition to the reception signal data.

### Step S505: Process of Generating Display Information

In this process, the display information generation unit 1007 of the information processing unit 1000 generates display information that can be displayed on the displaying unit 1008 based on the reconstruction data acquired in step S504. Then, the display information generation unit 1007 transmits the generated display information to the displaying unit 1008.

### Step S506: Process of Displaying Image

In this process, the displaying unit 1008 displays an image of the reconstruction data relating to the subject information based on the display information received from the display information generation unit 1007. The display information generation unit 1007 can cause the displaying unit 1008 to display distribution information or numerical information of the reconstruction data relating to the subject information.

For example, if the reconstruction data is displayed by MPR (Multi Planner Reconstruction), a cross-sectional image of the reconstruction data and a boundary of a region divided depending on the image quality on the cross-sectional image are displayed in a superimposed manner. Also, a display image may be displayed by volume rendering. Also, pixel values at respective positions of three-dimensional reconstruction data, that is, explanation by text based on voxel values of volume data may be displayed. Also, the display information generation unit 1007 may set a desirable display method by an instruction from the user as long as the display information relates to the reconstruction data.

By executing the above-described operations, subject information with high S/N and high resolution in the imaging region can be acquired.

Alternatively, reconstruction data may be acquired from signal information of a photoacoustic wave every pulse of light, and final reconstruction data may be acquired by combining the reconstruction data of each pulse. In particular, by acquiring reconstruction data for each pulse in a period between pulses, the period of time from when the measurement of photoacoustic waves is finished to when final reconstruction data is acquired can be decreased.

Also, in this embodiment, the example has been described in which the photoacoustic wave is measured while the probe 103 is moved in the XY directions. However, if the size of the imaging region 102 is small and the imaging region 102 is arranged in the measurement region 108, the probe 103 may not be moved.

Also, in this embodiment, description has been given with the example including the process in which the user designates a desirable imaging region. However, setting of a measurement position in this embodiment may be applied to a predetermined imaging region. For example, the imaging information acquisition unit 1001 may set the inside of the holding unit 1111, the shape of which is previously known, may be set as an imaging region. Also, if a plurality of holding units with different shapes are used, information of a plurality of imaging regions corresponding to the plurality of holding units can be saved in the data recording unit 1006. Then, the imaging information acquisition unit 1001 reads out the type of the holding unit, and reads out information relating to a corresponding imaging region from the data recording unit 1006, so that the imaging region can be set.

Also, setting of a measurement position according to this embodiment and setting of a measurement position so as to fill the imaging region with the high-resolution region with a priority given to the decrease in reconstruction artifact may be selectively switched. That is, the photoacoustic apparatus according to this embodiment may provide switching between the movement of the probe 103 regarding the measurement region, and the movement of the probe 103 regarding the high-resolution region in which the resolution isotropically changes. In this case, in step S501, any of setting of the measurement position regarding the measurement region and setting of the measurement position regarding the high-resolution region in which the resolution isotropically changes may be input as the imaging instruction information by the input unit 306.

### Second Embodiment

In the first embodiment, the case in which a photoacoustic wave is measured while the probe 103 is two-dimensionally moved in the in-plane direction (XY directions) of the opening of the probe 103 has been described. In contrast, in a second embodiment, a case in which a photoacoustic wave is measured while the probe 103 is three-dimensionally moved is described. That is, in this embodiment, a photoacoustic wave is measured while the probe 103 is moved in not only the XY directions but also the Z direction during a single shot of image taking.

The same reference signs are basically applied to the same components as those of the first embodiment, and the redundant description is omitted.

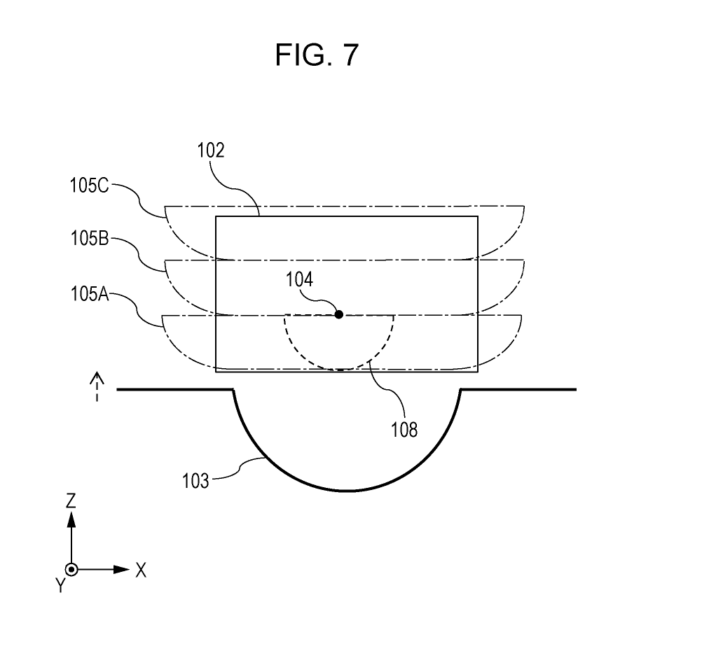

FIG. 7 is an illustration showing an imaging region and a locus of a measurement region according to this embodiment. In this embodiment, it is assumed that the measurement region 108 is a hemispherical region near the probe 103 of a sphere centered on the curvature center 104 of the probe 103 similarly to the first embodiment.

In this embodiment, the signal measurement unit 1100 performs measurement so that loci 105A, 105B, and 105C of the measurement region fill the entire region of the imaging region 102.

First, the measurement method determination unit 1003 sets a measurement position so that an end portion near the probe 103 of the measurement region 108 is aligned with an end portion of the imaging region 102. Then, based on the set measurement position, the moving unit 1102 moves the probe 103, and the light source 1104 emits light at a predetermined time point. Accordingly, a reception signal of a photoacoustic wave that allows acquisition of reconstruction data with high resolution of the locus 105A of the measurement region can be acquired.

Also, as shown in FIG. 7, if the size in the Z direction of the imaging region 102 is smaller than the size in the Z direction of the measurement region 108, the position of the probe 103 in the Z direction is changed. Also, a photoacoustic wave is measured in the XY directions similarly, and the locus 105B of the measurement region is formed. Then, the position in the Z direction of the probe 103 is further changed and a photoacoustic wave is measured in the XY directions. Hence, the locus 105C is formed. As shown in FIG. 7, by measuring a photoacoustic wave in the XY directions in order from the lower end of the imaging region 102, the imaging region 102 can be filled with the hemispherical region near the probe 103 centered on the curvature center 104 with a high priority. Even when the probe 103 is three-dimensionally moved, measurement may be performed such that an end portion near the probe 103 of the locus of the measurement region is aligned with an end portion near the probe 103 of the imaging region 102.

In this embodiment, measurement is performed so that the loci 105A to 105C of the measurement region do not overlap each other. However, as long as the loci of the measurement region can fill the imaging region, any measurement may be performed. That is, the loci of the measurement region formed by two-dimensional movement of the probe 103 may overlap each other.

Also, the pitch of the measurement position in the out-plane direction (Z direction) of the opening of the probe 103 may be smaller than the pitch of the measurement position in the in-plane direction (XY directions) of the opening of the probe 103. That is, the moving amount in the Z direction may be smaller than the moving amount in the XY directions during an intermission of light irradiation. With regard to attenuation in photoacoustic wave, since the change in resolution in the Z direction is more rapid than that in the XY directions, the variation in resolution can be decreased by a limited number of measurements by such a measurement.

Also, when the probe 103 is three-dimensionally moved, any moving method may be employed without limiting to the moving method of this embodiment. For example, a photoacoustic wave may be measured while the probe 103 is moved in all directions of X, Y, and Z during an intermission of light irradiation.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of U.S. Patent Application No. 62/028,571, filed Jul. 24, 2014, which is hereby incorporated by reference herein in its entirety.

What is claimed is:

1. A photoacoustic apparatus, comprising:
a light source;
a probe including
a plurality of transducers each configured to receive a photoacoustic wave generated from a subject irradiated with light emitted from the light source and output a reception signal, and
a support member having an opening and configured to support the plurality of transducers so that directivity axes of the plurality of transducers are collected;
a moving unit configured to two-dimensionally move the probe in an in-plane direction of the opening;
a region setting unit configured to set an imaging region; and
a processing unit configured to acquire subject information in the imaging region based on the reception signals output from the plurality of transducers,
wherein the light source is configured to emit the light if a position at which the directivity axes are collected is farther from the probe than a center of the imaging region.

2. The photoacoustic apparatus according to claim 1, wherein the light source is configured to emit the light if the position at which the directivity axes are collected is included in the imaging region.

3. The photoacoustic apparatus according to claim 1, wherein the light source is configured to emit the light if an end portion near the probe of a sphere centered on the position at which the directivity axes are collected is aligned with an end portion near the probe of the imaging region.

4. The photoacoustic apparatus according to claim 3,
wherein the support member has a shape based on a sphere,
wherein the position at which the directivity axes are collected is a curvature center of the support member, and
wherein a radius r of the sphere centered on the position at which the directivity axes are collected is determined by an expression as follows,
$${d}_{th} = \frac{{r}_{0}}{{φ}_{d}} \cdot R$$
where r₀ is a radius of the support member, and φ_{d} is a diameter of the transducers, R is a lower-limit resolution.

5. A photoacoustic apparatus, comprising:
a light source;
a probe including
a plurality of transducers each configured to receive a photoacoustic wave generated from a subject irradiated with light emitted from the light source and output a reception signal, and
a support member configured to support the plurality of transducers so that directivity axes of the plurality of transducers are collected;
a moving unit configured to move the probe;
a region setting unit configured to set an imaging region; and
a processing unit configured to acquire subject information in the imaging region based on the reception signals output from the plurality of transducers,
wherein the light source is configured to emit the light at a plurality of time points, and
wherein the moving unit is configured to move the probe so that a locus of a region near the probe of a sphere centered on a position at which the directivity axes are collected at the plurality of respective time points fills the imaging region.

6. The photoacoustic apparatus according to claim 5, wherein the moving unit is configured to move the probe so that a locus of a hemispherical region near the probe of the sphere centered on the position at which the directivity axes are collected at the plurality of respective time points fills the imaging region.

7. The photoacoustic apparatus according to claim 6, wherein the light source is configured to emit the light if the position at which the directivity axes are collected is included in the imaging region at each of the plurality of time points.

8. The photoacoustic apparatus according to claim 5, wherein the moving unit is configured to three-dimensionally move the probe.

9. The photoacoustic apparatus according to claim 8,
wherein the support member has an opening, and
wherein the moving unit is configured to cause a moving amount of the probe in an out-plane direction of the opening to be smaller than a moving amount in an in-plane direction of the opening during an intermission of the light irradiation.

10. The photoacoustic apparatus according to claim 5,
wherein the support member has a shape based on a sphere,
wherein the position at which the directivity axes are collected is a curvature center of the support member, and
wherein a radius r of the sphere centered on the position at which the directivity axes are collected is determined by an expression as follows,
$${d}_{th} = \frac{{r}_{0}}{{φ}_{d}} \cdot R$$
where r₀ is a radius of the support member, and φ_{d} is a diameter of the transducers, R is a lower-limit resolution.

11. The photoacoustic apparatus according to claim 10, wherein the lower-limit resolution is a value that is a half of the resolution at the curvature center of the support member.

12. The photoacoustic apparatus according to claim 1, further comprising:
an input unit configured to allow information relating to the imaging region to be input,
wherein the region setting unit is configured to set the imaging region based on the information relating to the imaging region input by the input unit.

13. The photoacoustic apparatus according to claim 1, further comprising:
a holding unit configured to hold the subject,
wherein the region setting unit is configured to set an inside of the holding unit as the imaging region.

14. The photoacoustic apparatus according to claim 1, wherein the support member has a shape being a hemispherical shape.

15. The photoacoustic apparatus according to claim 4, wherein the lower-limit resolution is a value that is a half of the resolution at the curvature center of the support member.

16. The photoacoustic apparatus according to claim 5, further comprising:
an input unit configured to allow information relating to the imaging region to be input,
wherein the region setting unit is configured to set the imaging region based on the information relating to the imaging region input by the input unit.

17. The photoacoustic apparatus according to claim 5, further comprising:
a holding unit configured to hold the subject,
wherein the region setting unit is configured to set an inside of the holding unit as the imaging region.

18. The photoacoustic apparatus according to claim 5, wherein the support member has a shape being a hemispherical shape.
